(19) United States
(12) Patent Application Publication
(10) Pub. No.: US 2008/0166712 A1
(43) Pub. Date: Jul. 10, 2008

(54) Gene detection method

(76) Inventor: Ryusuke Murayama, Ehime (JP)

Correspondence Address: WENDEROTH, LIND & PONACK L.L.P., 2033 K. STREET, NW, SUITE 800, WASHINGTON, DC, 20006, US

(21) Appl. No.: 11/783,572

(22) Filed: Apr. 10, 2007

(30) Foreign Application Priority Data
Apr. 10, 2006 (JP) 2006-107364

(51) Int. Cl. C12Q 1/68

(52) U.S. Cl. 435/6

(57) ABSTRACT

A gene detection method comprises an immobilization step of forming a single-stranded capture probe having a base sequence that is complementary to the target gene to be detected, and immobilizing the capture probe to a solid phase; a gene sample formation step of forming a gene sample by denaturing the target gene into a single strand; a bonding step of adding an electrochemically active substance to the gene sample to chemical bond's the gene sample with the electrochemically active substance; a gene sample capturing process of hybridizing the gene sample to which the electrochemically active substance is bonded, with the single-stranded capture probe that is immobilized to the solid phase, thereby to make the solid phase capture the gene sample; and a detection step of detecting the electrochemically active substance that is bonded to the gene sample immobilized to the solid phase, by electrochemical measurement.

### FIELD OF THE INVENTION

The present invention relates to a gene detection method for detecting a specific gene sequence that exists in a sample, with high sensitivity.

### BACKGROUND OF THE INVENTION

Conventionally, as a method for electrochemically detecting a specific gene sequence, there is a method using a DNA chip in which a single-stranded nucleic acid probe having a base sequence that is complementary to a target gene to be detected is immobilized on an electrode surface. In this method, the nucleic acid probe and the target gene sample that is denatured to a single strand are hybridized, and thereafter, a labeling agent which is electrochemically active and specifically binds to a double-stranded nucleic acid that is formed of the nucleic acid probe and the target gene sample is added to a reaction system for the nucleic acid probe and the gene sample, and then the labeling agent bonded to the double-stranded nucleic acid is detected by performing electrochemical measurement via the electrode, whereby the nucleic acid probe that is hybridized with the target gene sample is detected to confirm existence of the target gene (for example, refer to Japanese Published Patent Application No. Hei.5-199898 (Patent Document 1), and Japanese Published Patent Application No. Hei.9-288080 (Patent Document 2)).

The labeling agent indicates a substance that recognizes the double-stranded nucleic acid and specifically binds to the double-stranded nucleic acid. The labeling agent has a tabular intercalation base such as phenyl in a molecule, and binds to the double-stranded nucleic acid by that the intercalation base is intercalated between a base pair and a base pair of the double-stranded nucleic acid. The binding between the labeling agent and the double-stranded nucleic acid is a binding in an electrostatic interaction or a hydrophobic interaction, and it is a binding caused by an equilibrium reaction in which intercalation of the labeling agent into between the base pairs of the double-stranded nucleic acid and separation of the labeling agent from between the base pairs are repeated at a constant speed.

Furthermore, as the above-mentioned labeling agent, there is a substance that causes an electrically reversible oxidation-reduction reaction. When using such intercalator that causes an electrochemically reversible oxidation-reduction reaction, it is possible to detect existence of the labeling agent bonded to the double-stranded nucleic acid by measuring the electrochemical change. As an output signal of this electrochemical change, there is a current or a luminescence that occurs during the oxidation-reduction.

Accordingly, in the conventional gene detection method, it is important to specifically bond the labeling agent to only the double-stranded nucleic acid, and accurately detect the amount of the labeling agent bonded to the double-stranded nucleic acid.

However, the labeling agent is nonspecifically adsorbed to the single-stranded nucleic acid probe and to the electrode surface on which the nucleic acid probe is immobilized. The nonspecifically adsorbed labeling agent becomes a background noise when detecting the amount of the labeling agent bonded to the double-stranded nucleic acid, leading to a reduction in detection sensitivity.

In order to solve this problem, there is proposed a method for detecting existence of a target gene, which method comprises hybridizing a capture probe that is immobilized to a carrier such as beads, a labeling probe that labels an electrochemically active substance, and a gene sample, applying a voltage, and detecting an electrochemical signal from the labeling probe bonded to the gene sample (refer to Japanese Published Patent Application No. 2002-34561 (Patent Document 3)).

This method utilizes so-called sandwich hybridization. Since the capture probe, the gene sample, and the labeling probe are hybridized through specific interactions of the respective components, it provides high specificity, and enhances detection sensitivity.

In the method described in Patent Document 3, however, only one labeling probe is hybridized with one gene sample, and further, the labeling probe has only one or two electrochemically active substances labeled at an end of the single-stranded nucleic acid, and therefore, the signal intensity from the electrochemically active substance is low relative to the methods using the labeling agents as described in Patent Documents 1 and 2. Therefore, if the gene sample as a detection target has a low concentration, the gene sample must be amplified by a PCR or the like to increase the concentration thereof.

Moreover, there is another problem that the labeling probe must be prepared for each sample in addition to the capture probe.

### SUMMARY OF THE INVENTION

The present invention is made to solve the above-described problems and has for its object to provide a gene detection method which can detect a gene sample as a detection target with high sensitivity.

In order to solve the above-mentioned problems, according to the present invention, there is provided a gene detection method for detecting a gene having a specific sequence in a test sample, which method comprises an immobilization step of forming a single-stranded capture probe having a base sequence that is complementary to the target gene to be detected, and immobilizing the capture probe to a solid phase; a gene sample formation step of forming a gene sample by denaturing the target gene into a single strand; a bonding step of chemical bonding the gene sample and an electrochemically active substance; a gene sample capturing process of hybridizing the gene sample to which the electrochemically active substance is bonded with the single-stranded capture probe that is immobilized to the solid phase, thereby to make the solid phase capture the gene sample; and a detection step of detecting the electrochemically active substance that is bonded to the gene sample immobilized to the solid phase, by electrochemical measurement.

Further, according to the present invention, there is provided a gene detection method for detecting a gene having a specific sequence in a test sample, which method comprises a gene sample formation step of forming a gene sample by denaturing the target gene to be detected into a single strand; a bonding step of chemical bonding the gene sample to a linker having a site that binds to an electrochemically active substance; a double-stranded nucleic acid formation step of forming a single-stranded capture probe having a base sequence that is complementary to the target gene, and hybridizing the capture probe with the gene sample to which the linker is bonded, thereby forming a double-stranded nucleic acid; a reaction step of chemical bonding the electrochemically active substance and the linker of the gene sample in which the double-stranded nucleic acid is formed; an immobilization step of immobilizing the capture probe in which the double-stranded nucleic acid is formed, to a solid phase; and a detection step of detecting the electrochemically active substance that is bonded to the gene sample immobilized to the solid phase, by electrochemical measurement.

Further, according to the present invention, there is provided a gene detection method for detecting a gene having a specific sequence in a test sample, which method comprises an immobilization step of forming a single-stranded capture probe having a base sequence that is complementary to the target gene to be detected, and immobilizing the capture probe to a solid phase; a gene sample formation step of forming a gene sample by denaturing the target gene into a single strand; a bonding step of chemical bonding the gene sample and a linker having a site that binds to an electrochemically active substance; a gene sample capturing process of hybridizing the gene sample to which the linker is bonded with the single-stranded capture probe that is immobilized to the solid phase, thereby to make the solid phase capture the gene sample to which the linker is bonded; a reaction step of adding the electrochemically active substance to the gene sample that is captured by the solid phase, thereby chemical bonding the linker that is bonded to the gene sample, to the electrochemically active substance; and a detection step of detecting the electrochemically active substance that is bonded to the gene sample immobilized to the solid phase, by electrochemical measurement.

Further, according to the present invention, there is provided a gene detection method for detecting a gene having a specific sequence in a test sample, which method comprises a gene sample formation step of forming a gene sample by denaturing the target gene to be detected into a single strand; a bonding step of chemical bonding the gene sample and an electrochemically active substance; a double-stranded nucleic acid formation step of forming a single-stranded capture probe having a base sequence that is complementary to the target gene, and hybridizing the capture probe with the gene sample to which the electrochemically active substance is bonded, thereby forming a double-stranded nucleic acid; an immobilization step of immobilizing the capture probe in which the double-stranded nucleic acid is formed, to a solid phase; and a detection step of detecting the electrochemically active substance that is bonded to the gene sample immobilized to the solid phase, by electrochemical measurement.

Further, in the gene detection method according to the present invention, the bonding step of bonding the gene sample and the electrochemically active substance is carried out, after a halogen compound is added to bases in the gene sample, by promoting a nucleophilic substitution reaction between a functional group in the electrochemically active substance and the halogen that is bonded to the bases in the gene sample.

Further, in the gene detection method according to the present invention, the bonding step of bonding the gene sample and the linker is carried out, after a halogen compound is added to bases in the gene sample, by promoting a nucleophilic substitution reaction between a functional group in the linker and the halogen that is bonded to the bases in the gene sample.

Further, in the gene detection method according to the present invention, the electrochemically active substance is represented by chemical formula (1) as follows:

NuLa)m-E

wherein Nu is a nucleophile selected from among amine group, alcohol group, ether group, thiol group, and oxide group, E is an electrochemically active site, and La is a connection site that connects the Nu to the E.

Further, in the gene detection method according to the present invention, the linker is represented by chemical formula (2) as follows:

NuLb)n-Sa

wherein Nu is a nucleophile selected from among amine group, alcohol group, ether group, thiol group, and oxide group, Sa is a site that chemical bonds to the electrochemically active substance, and Lb is a connection site that connects the Nu to the Sa.

Further, in the gene detection method according to the present invention, the electrochemically active substance is represented by chemical formula (3) as follows:

ELc)o-Sb

wherein E is an electrochemically active site, Sb is a site that chemical bonds to the Sa, and Lc is a connection site that connects the Sb to the E.

Further, in the gene detection method according to the present invention, the La, Lb, and Lc are substances selected from among alkyl, alcohol, carboxylic acid, sulfo acid, ester, ketone, thiol, ether, amine, nitro, nitrile, sugar, phosphate acid, amino acid, methacrylic acid, amide, imide, isoprene, urethane, uronic acid, ethylene, carbonate, vinyl, cycloalkane, and heterocyclic compound, and a combination of some of these substances.

Further, in the gene detection method according to the present invention, the chemical bonding between the Sa and the Sb is one selected from among amide bonding, ester bonding, ether bonding, thioether bonding, sulfide bonding, carbonyl bonding, imino bonding, and antibody-antigen bonding.

Further, in the gene detection method according to the present invention, the m is an integer ranging from 4 to 50.

Further, in the gene detection method according to the present invention, the n is an integer ranging from 1 to 50.

Further, in the gene detection method according to the present invention, the o is an integer ranging from 1 to 1000.

Further, in the gene detection method according to the present invention, the o is an integer ranging from 3 to 1000 when the n is 1, and the o is an integer ranging from 2 to 1000 when the n is 2.

Further, in the gene detection method according to the present invention, the E is a compound having oxidation-reduction property.

Further, in the gene detection method according to the present invention, the compound having oxidation-reduction property is a compound which exhibits electrochemiluminescence.

Further, in the gene detection method according to the present invention, the compound which exhibits electrochemiluminescence is one selected from among a metal complex having a heterocyclic compound as a ligand, rubrene, anthracene, coronene, pyrene, fluoranthene, chrysene, phenanthrene, perylene, binaphthyl, and octatetraene.

Further, in the gene detection method according to the present invention, the metal complex having a heterocyclic compound as a ligand is a metal complex having a pyridine site as a ligand.

Further, in the gene detection method according to the present invention, the metal complex having a pyridine site as a ligand is one of a metal bipyridine complex and a metal phenanthroline complex.

Further, in the gene detection method according to the present invention, a center metal of the metal complex having a heterocyclic compound as a ligand is one of ruthenium and osnium.

Further, in the gene detection method according to the present invention, the detection step includes applying a voltage to the solid phase, and measuring the quantity of electrochemiluminescence from the linked electrochemically active substance.

### EFFECTS OF THE INVENTION

According to the gene detection method of the present invention, plural electrochemically active substances can be chemical bonded, directly or via a linker, to a gene sample to be detected, thereby achieving high sensitivity. Further, when the sequence of the gene to be detected is longer, a larger amount of the electrochemically active substances are bonded to the gene sample, and therefore, this method is also effective in detecting a raw sample. Furthermore, since no labeling probe is required, detection can be carried out easily and inexpensively.

### BRIEF DESCRIPTION OF DRAWINGS

FIG. 1 is a diagram illustrating measured integral electrochemiluminescence quantity according to Example 1 of the present invention.

FIG. 2 is a diagram illustrating measured integral electrochemiluminescence quantity according to Example 2 of the present invention.

FIG. 3 is a diagram illustrating measured integral electrochemiluminescence quantity according to Example 3 of the present invention.

### DETAILED DESCRIPTION OF PREFERRED EMBODIMENTS

Hereinafter, a gene detection method according to the present invention will be described in detail. In the following embodiments, a gene sample is obtained by disrupting cells in an arbitrary sample including such as blood, white blood cell, blood serum, urine, feces, semen, saliva, cultured cell, tissue cell such as cells of various organs, and other genes, to liberate a double-stranded nucleic acid from the sample, and then dissociating the double-stranded nucleic acid into a single-stranded nucleic acid by thermal treatment or alkali treatment. Further, the gene sample according to the embodiments of the present invention may be a nucleic acid segment that is cut off by a restriction enzyme and purified by such as separation using electrophoresis.

### Embodiment 1

Hereinafter, a gene detection method according to a first embodiment will be described.

### (Step 1)

Initially, a capture probe is formed. This capture probe has a sequence that is equal to a whole or a part of a sequence of a gene to be detected.

This capture probe may be a single-stranded nucleic acid obtained by chemical synthesis, or a nucleic acid which is obtained by cutting a nucleic acid extracted from a biologic sample with a restriction enzyme, and purifying the nucleic acid by separation due to electrophoresis or the like. In the case of using the nucleic acid extracted from the biologic sample, it is preferable to dissociate the nucleic acid into a single-stranded nucleic acid by thermal treatment or alkali treatment.

### (Step 2)

Thereafter, the capture probe obtained as described above is immobilized to a solid phase. A solid phase used in the present invention is not particularly restricted, and examples of the solid phase include a noble metal such as gold, platinum, platinum black, palladium, or rhodium, a carbon such as graphite, glassy carbon, pyrolytic graphite, carbon paste, or carbon fiber, an oxide such as titanic oxide, tin oxide, manganese oxide, or lead oxide, or a semiconductor such as Si, Ge, ZnO, CdS, TiO, or GaAs. These materials can be utilized as electrodes. In this case, these electrodes may be covered with a conductive polymer, whereby more stable capture probe immobilized electrodes can be prepared.

As a method for immobilizing the capture probe to the solid phase, a well-know method is adopted. For example, when the solid phase is a gold electrode, thiol group is introduced to a 5′- or 3′-terminal (preferably, 5′-terminal) of the capture probe to be immobilized, and the capture probe is immobilized to the gold electrode through covalent bonding between gold and sulfur. This method of introducing the thiol group to the capture probe is described in “M. Maeda et al., Chem. Lett., 1805 ˜1808 (1994)”, and “B. A. Connolly, Nucleic Acids Res., 13, 4484 (1985)”.

That is, the capture probe having the thiol group, which is obtained by the above-mentioned method, is dropped onto the gold electrode, and the gold electrode is left for a few hours under a low temperature, whereby the capture probe is immobilized onto the electrode, resulting in a capture probe.

As another example of the solid phase, particles having magnetism, which are generally called magnetic beads, may be adopted. In the case of using the magnetic beads, immobilization of the capture probe may be performed by an avidin-biotin bonding method. Initially, avidin is coated over the surface of the magnetic beads. On the other hand, biotin is bonded to an end of the capture probe. When this capture probe is added to the magnetic beads, an antibody-antigen reaction occurs, whereby the capture probe can be bonded to the magnetic beads. Since this method is well known, the detail will be omitted.

### (Step 3)

Next, a gene sample to be a detection target is formed. This gene sample is obtained by, as described above, disrupting cells in an arbitrary sample to liberate a double-stranded nucleic acid from the sample, and then dissociating the double-stranded nucleic acid into a single-stranded nucleic acid by thermal treatment or alkali treatment.

At this time, the disruption of the cells in the sample can be performed by an ordinary method, and for example, it can be performed by externally applying a physical function such as shaking or supersonic. Further, it is also possible to liberate a nucleic acid from cells by using a nucleic acid extraction solution (e.g., a surface-activating agent such as SDS, Triton-X, or Tween-20, or a solution including saponin, EDTA, or protease).

### (Step 4)

halogen compound is added to the bases of the single-stranded DNA thus obtained. This halogen compound is not especially restricted, and any halogen compound may be adopted so long as halogen can be added to the bases of the DNA. For example, chlorosuccinimido, bromosuccinimido, or iodosuccinimido may be adopted. An appropriate amount of this halogen compound solution is added to the above-mentioned single-stranded DNA, and a buffer solution such as sodium hydrogen carbonate is dropped. Thus obtained solution is subjected to gentle mixing for ten minutes by icing, whereby the halogen group can be bonded to the bases of the single-stranded DNA.

### (Step 5)

Next, a substance that is electrochemically active (hereinafter simply referred to as “an electrochemically active substance) is added to covalently combine the single-stranded DNA with the electrochemically active substance.

This electrochemically active substance has a functional group that performs a nucleophilic substitution reaction with the halogen group that is bonded to the bases of the single-stranded DNA, and it is represented by chemical formula (4) as follows.

NuLa)m-E

wherein Nu is a nucleophile agent selected from among amine group, alcohol group, ether group, thiol group, and oxide group, E is an electrochemically active site, and La is a connection site that connects the Nu and the E.

The La shown in chemical formula (4) is a substance selected from among alkyl, alcohol, carboxylic acid, sulfo acid, ester, ketone, thiol, ether, amine, nitro, nitrile, sugar, phosphate acid, amino acid, methacrylic acid, amide, imide, isoprene, urethane, uronic acid, ethylene, carbonate, vinyl, cycloalkane, and heterocyclic compound, or a combination of some of these substances.

Further, the m shown in chemical formula (4) is desired to be an integer from 4 to 50. The reason is as follows. If the m is smaller than 4, the space between the electrochemically active substance and the gene sample is narrow, and thereby it becomes difficult to form a double-stranded structure. Further, when the m is larger than 50, the electrochemically active substance itself becomes a steric hindrance, and furthermore, the nucleophilic substitution reaction with the halogen group that is bonded to the bases of the single-stranded DNA becomes difficult.

The E as an electrochemically active site is not especially restricted so long as it is an electrochemically detectable substance. For example, a compound having an oxidation-reduction property, which is detectable by measuring an oxidation-reduction current that occurs during a reversible oxidation-reduction reaction, may be adopted.

Examples of the compound having such oxidation-reduction property include ferrocene, catecholamine, a metal complex having a heterocyclic compound as a ligand, rubrene, anthracene, coronene, pyrene, fluoranthene, chrysene, phenanthrene, perylene, binaphthyl, octatetraene, and viologen.

Further, among the above-mentioned metal complex having a heterocyclic compound as a ligand, rubrene, anthracene, coronene, pyrene, fluoranthene, chrysene, phenanthrene, perylene, binaphthyl, and octatetraene, some of them generate electrochemiluminescence during the oxidation-reduction reaction, and the substance can be detected by measuring the luminescence.

Further, as the metal complex having a heterocyclic compound as a ligand, a heterocyclic compound including oxygen or nitrogen, e.g., a metal complex having a pyridine site or a pyran site as a ligand, may be adopted. Particularly, a metal complex having a pyridine site as a ligand is preferable.

Further, as the metal complex having a pyridine site as a ligand, a metal bipyridine complex or a metal phenanthroline complex may be adopted.

Furthermore, as a center metal in the metal complex having a heterocyclic compound as a ligand, there may be adopted ruthenium, osnium, zinc, cobalt, platinum, chrome, molybdenum, tungsten, technetium, rhenium, rhodium, iridium, palladium, copper, indium, lanthanum, praseodymium, neodymium, and samarium.

Particularly, a complex having ruthenium or osnium as a center metal has favorable electrochemiluminescent characteristic. As a material having such favorable electrochemiluminescent characteristic, ruthenium bipyridine complex, ruthenium phenanthroline complex, osnium bipyridine complex, or osnium phenanthroline complex may be adopted.

A specific example of the electrochemically active substance according to the first embodiment is shown by chemical formula (5) as follows.

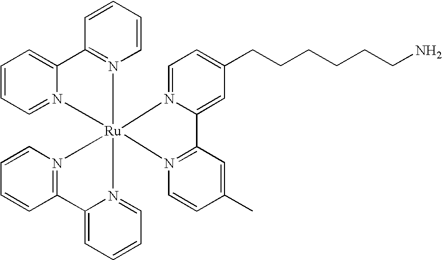

### (Step 6)

Next, the single-stranded DNA to which the electrochemically active substance is bonded (hereinafter referred to as “labeled gene sample”) is extracted. An extraction method is as follows. That is, only the DNA is precipitated by using ethanol or acetonitrile, and the solution is subjected to centrifugal separation, and then only the supernatant portion of the solution is removed. This process is repeated two or three times, and finally, the solution is displaced with a hybridized solution. As another method, the DNA may be purified with HPLC, or it may be separated by gel filtration chromatography. A DNA extraction kit that is commercially supplied by QIAGEN Co., Ltd. or the like may be used to achieve rapid extraction.

### (Step 7)

Thereafter, the solution including the labeled gene sample which is formed as described above is brought into contact with the capture probe that is immobilized to the solid phase. Thereby, the capture probe and the gene sample having a sequence that is complementary to the capture probe are hybridized, whereby the labeled gene sample is immobilized to the solid phase. Since a method for hybridizing the capture probe and the labeled gene sample is well known, the description will be omitted.

### (Step 8)

After a double-stranded DNA is formed by the capture probe and the labeled gene sample, it is washed with a phosphate buffer or the like to remove unreacted gene sample and the like.

As a result, existence of the double-stranded nucleic acid can be detected with high sensitivity by measuring an electrochemical signal from the electrochemically active substance that is chemical bonded to the hybridized double-stranded nucleic acid.

The electrochemical signal from the electrochemically active substance can be measured by a measurement system comprising a potentiostat, a function generator and the like when an electrochemically active substance that generates an oxidization-reduction current is used, although it depends on the type of the substance to be added. On the other hand, when an electrochemically active substance that generates electrochemical luminescence is used, an electrochemical signal can be measured by using a photomultiplier or the like.

While in this first embodiment the capture probe is immobilized to the solid phase and then hybridized with the labeled gene sample, the capture probe may be immobilized to the solid phase after it is hybridized with the labeled gene sample.

### Embodiment 2

While in the first embodiment the electrochemically active substance is directly applied to the single-stranded gene sample to which the halogen compound is added, bonding between the electrochemically active substance and the single-stranded gene sample to which the halogen compound is added may be performed through a linker.

### (Step 1)

Initially, a capture probe and a gene sample to be a detection target are formed. Since this process has been described in detail for the first embodiment, repeated description is not necessary.

### (Step 2)

Next, a halogen compound is added to the gene sample. Since this process has also been described in detail for the first embodiment, repeated description is not necessary.

### (Step 3)

Next, a linker is applied to the gene sample to which the halogen compound is added, thereby covalent bonding the single-stranded DNA with the linker.

This linker has a functional group that performs a nucleophilic substitution reaction with the halogen group that is bonded to the bases of the single-stranded DNA, and it is represented by chemical formula (6) as follows.

NuLb)n-Sa

wherein Nu is a nucleophile agent selected from among amine group, alcohol group, ether group, thiol group, and oxide group, Sa is a site that is chemical bonded to the electrochemically active substance, and Lb is a connection site that connects the Nu to the Sa.

The Lb shown in chemical formula (6) is a substance selected from among alkyl, alcohol, carboxylic acid, sulfo acid, ester, ketone, thiol, ether, amine, nitro, nitrile, sugar, phosphate, amino acid, methacrylic acid, amide, imide, isoprene, urethane, uronic acid, ethylene, carbonate, vinyl, cycloalkane, and heterocyclic compound, or a combination of some of these substances.

Further, Sa has a functional group that can be bonded to Sb included in an electrochemically active substance to be described later, by any of chemical bondings comprising amide bonding, ester bonding, ether bonding, thioether bonding, sulfide bonding, carbonyl bonding, imino bonding, and antibody-antigen bonding. Since the linker and the electrochemically active substance can be bonded by chemical bonding having a strong bonding force, strong washing can be carried out when removing unnecessary gene sample.

Further, the n shown in chemical formula (6) is desired to be an integer from 1 to 50. The reason is as follows. If the n is larger than 50, the electrochemically active substance itself becomes a steric hindrance, and furthermore, the nucleophilic substitution reaction with the halogen group that is bonded to the bases of the single-stranded DNA becomes difficult.

A specific example of the linker according to the second embodiment is represented by chemical formula (7) as follows.

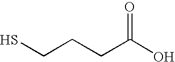

### (Step 4)

Next, the single-stranded DNA to which the linker is bonded (hereinafter referred to as “linker-bonded gene sample”) is extracted. An extraction method of as follows. That is, only the DNA is precipitated by using ethanol or acetonitrile, and the solution is subjected to centrifugal separation, and then only the supernatant portion of the solution is removed. This process is repeated two or three times, and finally, the solution is displaced with a hybridized solution. As another method, the DNA may be purified with HPLC, or it may be separated by gel filtration chromatography. A DNA extraction kit that is commercially supplied by QIAGEN Co., Ltd. or the like may be used to achieve rapid extraction.

### (Step 5)

The linker-bonded gene sample thus obtained is brought into contact with the above-mentioned capture probe. Thereby, the capture probe and the linker-bonded gene sample are hybridized. Since the method for hybridizing the capture probe and the linker-bonded gene sample is well known, the description thereof will be omitted.

### (Step 6)

Next, an electrochemically active substance is applied to the linker-bonded gene sample which is hybridized with the capture probe to produce a labeled gene sample.

This electrochemically active substance has a functional group that is chemical bonded to the above-mentioned linker, and is represented by chemical formula (8) as follows.

ELC)o-Sb

wherein E is an electrochemically active site, Sb is a site that chemical binds to the Sa, and Lc is a connection site that connects the Sb to the E.

The Lc shown in chemical formula (8) is a substance selected from among alkyl, alcohol, carboxylic acid, sulfo acid, ester, ketone, thiol, ether, amine, nitro, nitrile, sugar, phosphate, amino acid, methacrylic acid, amide, imide, isoprene, urethane, uronic acid, ethylene, carbonate, vinyl, cycloalkane, and heterocyclic compound, or a combination of some of these substances.

Further, the Sb has a functional group that can be bonded to the Sa included in the above-mentioned linker by any of chemical bondings comprising amide bonding, ester bonding, ether bonding, thioether bonding, sulfide bonding, carbonyl bonding, imino bonding, and antibody-antigen bonding. Thereby, since the linker and the electrochemically active substance can be bonded by chemical bonding having a strong bonding force, strong washing can be carried out when removing unnecessary gene sample.

Further, the o shown in chemical formula (8) is desired to be an integer ranging from 1 to 1000 (however, the o is an integer from 3 to 1000 when the n is equal to 1, and it is an integer from 2 to 1000 wen the n is equal to 2). The reason is as follows. If the o is larger than 1000, chemical bonding between the electrochemically active substance and the linker-bonded gene sample becomes difficult due to steric hindrance. Further, when the n is 1, the o is desired to be an integer ranging from 3 to 1000. The reason is as follows. If the o is not larger than 2, reaction between the gene sample and the electrochemically active substance becomes impossible due to steric hindrance. Further, when the n is 2, the o is desired to be an integer ranging from 2 to 1000. The reason is as follows. If the o is 1, reaction between the gene sample and the electrochemically active substance becomes impossible due to steric hindrance.

Any substance may be adopted for the E as the electrochemically active site so long as it is electrochemically detectable substance. For example, a compound having an oxidation-reduction property, which is detectable by measuring an oxidation-reduction current that occurs during reversible oxidation-reduction reaction, may be adopted.

Examples of compounds having such oxidation-reduction property include ferrocene, catecholamine, a metal complex having a heterocyclic compound as a ligand, rubrene, anthracene, coronene, pyrene, fluoranthene, chrysene, phenanthrene, perylene, binaphthyl, octatetraene, and viologen.

Further, among the above-mentioned metal complex having a heterocyclic compound as a ligand, rubrene, anthracene, coronene, pyrene, fluoranthene, chrysene, phenanthrene, perylene, binaphthyl, and octatetraene, some of them generate electrochemiluminescence during oxidation-reduction reaction, and the substance can be detected by measuring this luminescence.

Further, as the metal complex having a heterocyclic compound as a ligand, a heterocyclic compound including oxygen or nitrogen, for example, a metal complex having a pyridine site or a pyran site as a ligand, may be adopted. Particularly, a metal complex having a pyridine site as a ligand is preferable.

Furthermore, as the metal complex having a pyridine site as a ligand, a metal bipyridine complex or a metal phenanthroline complex may be adopted.

Furthermore, as a center metal in the metal complex having a heterocyclic compound as a ligand, there may be adopted ruthenium, osnium, zinc, cobalt, platinum, chrome, molybdenum, tungsten, technetium, rhenium, rhodium, iridium, palladium, copper, indium, lanthanum, praseodymium, neodymium, and samarium.

Particularly, a complex having ruthenium or osnium as a center metal has favorable electrochemiluminescent characteristic, and examples of materials having such favorable electrochemiluminescent characteristic include ruthenium bipyridine complex, ruthenium phenanthroline complex, osnium bipyridine complex, and osnium phenanthroline complex.

A specific example of the electrochemically active substance according to the second embodiment is represented by chemical formula (9) as follows.

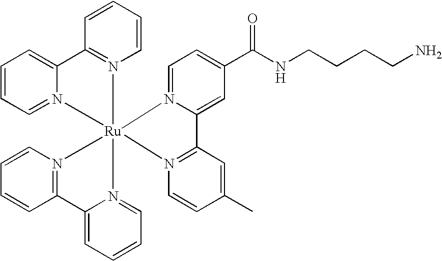

### (Step 7)

After the electrochemically active substance is bonded to the linker-bonded gene sample, it is immobilized to a solid phase. Since this method has already been described in detail for the first embodiment, repeated description is not necessary.

### (Step 8)

After the immobilization, the sample is washed with a phosphate buffer or the like to remove the nonspecifically adhered gene sample or the like.

As a result, existence of the double-stranded nucleic acid can be detected with high sensitivity by measuring an electrochemical signal from the electrochemically active substance that is chemical bonded to the hybridized double-stranded nucleic acid. Since the measurement method has described in detail for the first embodiment, repeated description is not necessary.

While in this second embodiment, the capture probe is immobilized to the solid phase after it is hybridized with the linker-bonded gene sample and the electrochemically active substance is added, the linker-bonded gene sample and the capture probe may be hybridized after the capture probe is immobilized to the solid phase.

Further, while in this second embodiment the electrochemically active substance is added after the capture probe and the linker-bonded gene sample are hybridized, it may be added simultaneously with the hybridization. At this time, the o is desired to be an integer ranging from 1 to 50. The reason is as follows. If the o is larger than 50, it might adversely affect the hybridization.

### Example 1

Hereinafter, an example of the present invention will be described, but the present invention is not restricted thereto.

### (1) Immobilization of Nucleic Acid Probe to Solid Phase Surface

A gold electrode is used as a solid phase. This gold electrode is prepared by depositing 200 nm thick gold with 10 nm thick titanium as a base layer, on a glass substrate, using a sputtering apparatus (SH-350 produced by UlVAC, Inc.), and then forming an electrode pattern in a photolithography process. Further, the electrode surface is washed for one minute with piranha solution (hydrogen peroxide:concentrated sulfuric acid=1:3), and rinsed with pure water, and then dried by nitrogen blow.

Employed as a capture probe is 30-base oligodeoxynucleotide which is modified with thiol group via 5′-terminal phosphate group, and has a sequence of AATTTGTTATGGGTTCCCGG GAAATAATCA (sequence number 1) from the 5′-terminal.

Then, the capture probe is dissolved in 10 mM of PBS (a sodium phosphate buffer solution of pH 7.4) to adjust it to 10M.

Thus adjusted capture probe solution is dropped onto the gold electrode, and the gold electrode is left for twelve hours at room temperature under saturated humidity, whereby the thiol group and the gold are bonded to each other to immobilize the capture probe to the gold electrode.

### (2) Modification of Electrochemically Active Substance to Gene Sample

Employed as a gene sample is 100-base oligodeoxynucleotide having a sequence of AATTGAATGA AAACATCAGG ATTGTAAGCA CCCCCTGGAT CCAGATATGC AATAATTTTC CCACTATCAT TGATTATTTC CCGGGAACCC ATAACAAATT (sequence number 2), which is positioned at 599-698th from a 5′-terminal of a gene sequence of human origin Cytochrome P-450.

The gene sample adjusted to 100 μM is collected by 100 μL, and N-bromosuccinimide adjusted to 2 μM is added by 37.5 μL, and the solution is gently stirred for 5 minutes while cooling the same with ice water.

After the agitation, the electrochemically active substance (chemical formula (10)) adjusted to 1 mM is added by 100 μL.

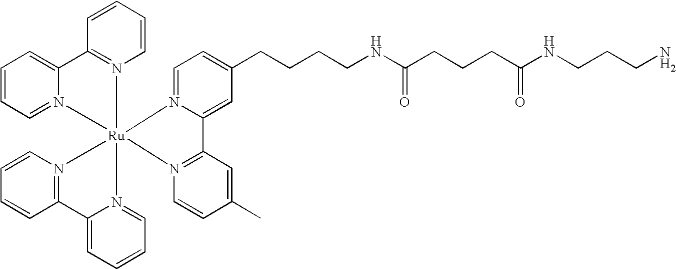

The electrochemically active substance that is represented by chemical formula (10) is obtained as follows.

Initially, 2.50 g (13.5 mmol) of 4,4′-dimethyl-2,2′bipyridine solution which is dissolved in 60.0 mL of tetrahydrofuran THF) is injected into a container under nitrogen atmosphere, and thereafter, 16.9 mL (27.0 mmol) of lithium diisopropylamide 2M solution is dropped, and the solution is stirred for 30 minutes while cooling the same. On the other hand, 4.2 mL (41.1 mmol) of 1,3-dibromopropane and 10 mL of THF are added in a container that is similarly dried in nitrogen gas stream, and the solution is stirred while cooling the same. The above-mentioned reaction solution is slowly dropped into this container, and reaction is promoted for 2.5 hours. The reaction solution is neutralized with 2N of hydrochloric acid, and the THF is distilled, and thereafter, the reactant is extracted with chloroform. Further, the crude product obtained by distilling the solvent is purified with silica gel column to obtain a product C (yield 47%).

Then, 11.0 g (3.28 mmol) of the product C, 0.67 g (3.61 mmol) of phthalimide potassium, and 30.0 mL of dimethylformamide (dehydrated) are added in a container under nitrogen atmosphere, and the solution is refluxed for eighteen hours in an oil bath. After reaction, the reactant is extracted with chloroform, and washed with distilled water using 50 mL of 0.2N sodium hydroxide. The solvent is distilled away, and recrystallization is performed by ethyl acetate and hexane, thereby obtaining a product D (yield 61.5%).

After ruthenium chloride (III) (2.98 g, 0.01 mol) and 2,2′-bipyridyl (3.44 g, 0.022 mol) are refluxed for six hours in dimethylformamide (80.0 mL), the solvent is distilled away. Thereafter, acetone is added, and the solution is cooled overnight to obtain a black precipitation. Thus obtained black precipitation is extracted, and 170 mL of ethanol aqueous solution (ethanol:water=1:1) is added, and the solution is heated and refluxed for one hour. After filtration, 20 g of lithium chloride is added, and ethanol is distilled away, and further, the solution is cooled overnight. The deposited black substance is collected by suction filtration, thereby obtaining a product E (yield 68.2%).

Then, 0.50 g (1.35 mmol) of the product D, 0.78 g (1.61 mmol) of the product E, and 50 mL of ethanol are added in a container that is nitrogen substituted. After this solution is refluxed for nine hours under nitrogen atmosphere, the solvent is distilled away, and the resultant is dissolved with distilled water and precipitated in 1.0M of perchloric acid solution. This precipitate is collected, and recrystallization is carried out with methanol, thereby obtaining a product F (yield 81.6%).

Furthermore, 11.0 g (1.02 mmol) of the product F and 70.0 mL of methanol are refluxed for one hour. After the solution is cooled down to room temperature, 0.21 mL (4.21 mmol) of hydrazine monohydrate is added, and the solution is again refluxed for thirteen hours. After reaction, 15 mL of distilled water is added, and methanol is distilled away.

Next, a reaction solution that is obtained by adding 5.0 mL of concentrated hydrochloric acid and performing refluxing for two hours is cold-stored overnight, and impurities are removed by normal filtration.

After this solution is neutralized with sodium hydrogen carbonate, water is distilled away, and inorganic substances are removed with acetonitrile. The crude product obtained by distilling the solvent away is purified with silica gel column, thereby obtaining a product G (yield 71.4%).

Then, 0.65 g (0.76 mmol) of the product G is added in a container that is shaded by aluminum foil, and dissolved in 10 mL of acetonitrile. Next, 0.23 g (2.29 mmol) of triethylamine is added, and thereafter, 0.87 g (7.62 mmol) of glutaric anhydride dissolved in 20 mL of acetonitrile is dropped.

After promoting reaction for nine hours, a crude product that is obtained by distilling acetonitrile with an evaporator is purified by high performance liquid chromatography (HPLC), thereby obtaining a product H (yield 62.6%).

Then, 0.080 g (83 nmol) of this product H is stirred in 5.0 mL of acetonitrile, 0.052 g (0.24 mmol) of DCC is added, and this solution is stirred for four hours at room temperature. Therefore, 700/L (8.30 mmol) of 1,3-diamonopropane is dropped, and the solution is stirred for more two hours. The crude product is purified with silica gel column, thereby obtaining an electrochemically active substance represented by chemical formula (10) (yield 57.4%).

The following table shows the result of ¹H-NMR of the substance obtained as described above, which is represented by chemical formula (10).

¹H-NMR (300 MHz, DMSO d-6)
σ:

1.46 (2H, m)
1.70 (6H, m)
2.12 (4H, m)
2.54 (3H, s)
2.80 (2H, t)
2.85 (2H, t)
3.10 (4H, m)
3.42 (2H, bs)
7.39 (2H, t)
7.57 (6H, m)
7.76 (4H, m)
8.20 (2H, t)
8.76 (4H, t)
8.86 (6H, m)

After the gene sample is modified with the electrochemically active substance as described above, unreacted electrochemically active substance is removed using HPLC, and the solution is distilled by a centrifugal drying machine.

Upon confirming the number of bonds using a spectrophotometer, it is discovered that six molecules of the electrochemically active substance are bonded to one molecule of the gene sample.

### (3) Hybridization

The labeled gene sample obtained as described above is dissolved in 2×SSC and adjusted to 2.0 μM.

Thus prepared labeled gene sample is dropped onto the gold electrode to which the capture probe is fixed, and reaction is promoted for ten hours in a constant-temperature bath of 70° C. Thereby, an electrode x on which the labeled gene sample and the capture probe are hybridized and a double-stranded nucleic acid is formed is obtained.

Furthermore, in this first example, a capture probe having a sequence that is non-complementary to the gene sample is prepared as a comparison target, and the same processing performed for the above-mentioned nucleic acid probe is performed to this capture probe, thereby obtaining an electrode y on which the labeled gene sample and the capture probe are not hybridized and no double-stranded nucleic acid is formed. In this first embodiment, employed as the non-complementary capture probe is a probe which comprises 30 mer of Poly-A, has a sequence of AAAAAAAAAA AAAAAAAAAA AAAAAAAAAA (sequence number 3), and is modified with thiol group via 5′-terminal phosphate group.

### (4) Electrochemical Measurement

After the above-mentioned processes, 80 μL of an electrolytic solution in which 0.1M of PBS and 0.1M of triethylamine are mixed is dropped to the electrode x on which the double-stranded nucleic acid is formed and to the electrode y on which no double-stranded nucleic acid is formed, respectively. Thereafter, voltage is applied to the respective electrodes x and y, and electrochemiluminescence which occurs at this time is measured. This voltage application is carried out by scanning from 0V to 1.3V, and electrochemical measurement is carried out for three seconds. The measurement of electrochemiluminescence quantity is carried out using a photoelectron multiplier (H7360-01 produced by Hamamatsu Photonics), and the luminescence quantities obtained during the voltage scanning are integrated.

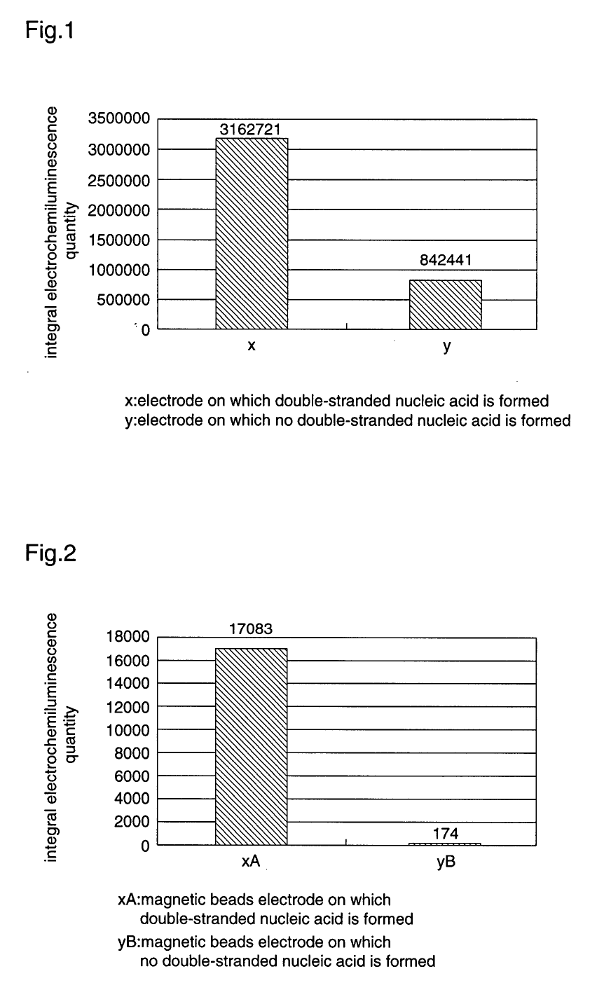

FIG. 1 is a diagram illustrating the electrochemiluminescence integral quantities which are detected on the electrode x having the double-stranded nucleic acid and on the electrode y having no double-stranded nucleic acid.

As is evident from FIG. 1, the luminescence quantity on the electrode x having the double-stranded nucleic acid is significantly larger than the luminescence quantity on the electrode y having no double-stranded nucleic acid, and it is discovered that the double-stranded nucleic acid, i.e., the target gene sample, can be detected with high sensitivity by using the method of the present invention.

### Example 2

### (1) Immobilization of Capture Probe to Solid Phase Surface

In this second example, magnetic beads are used as a solid phase. As the magnetic beads, CM01N/5896 streptavidin magnetic beads (particle diameter: 0.35 μm) produced by Bangs Laboratories Inc. are adopted. As a capture probe, a probe which is modified with biotin via 5′-terminal phosphate group, and has the same sequence as that of the first embodiment.

Initially, 1 mg of magnetic beads are collected, and washed with a TTL buffer (which is prepared so as to have a volume ratio of 2:10:5:3 for 500 mM Tris-HCl (pH 8.0):Tween20:2M lithium chloride:ultrapure water), and then displaced in 20 μL of TTL buffer. Thereafter, 100 μM of capture probe is added by 5 μL, and the solution is gently mixed for fifteen minutes at room temperature.

Then, the solution is decanted, and the remaining magnetic beads are washed with 0.15M of sodium hydroxide solution, and then washed with a TT buffer (which is prepared so as to have a volume ratio of 1:2:1 for 500 mM Tris-HCl (pH8.0):Tween20:ultrapure water).

After the washing, the solution is displaced in a TTE buffer, and incubated for ten minutes at 80° C. to remove unstable bonds. Thereby, the magnetic beads to which the capture probe is immobilized are obtained.

Further, in this second example, a capture probe having a sequence that is non-complementary to the gene sample is prepared as a comparison target, and the same processing performed for the above-mentioned nucleic acid probe is performed on the capture probe. In this second example, employed as the non-complementary capture probe is a probe which comprises 30 mer of Poly-A, has a sequence of AAAAAAAAAA AAAAAAAAAA AAAAAAAAAA (sequence number 3), and is modified with thiol group via 5′-terminal phosphate group.

### (2) Hybridization

The same substance as adopted in the first example is used for the labeled gene sample.

Then, 14 μL of 2×SSC is added to the magnetic beads to which the capture probe is immobilized, and 4 μL of the gene sample that is adjusted to 10 nM is applied, and the solution is gently mixed at 70° C. After agitation for ten hours, the solution is decanted, and washed with 2×SSC heated to 40° C., thereby obtaining magnetic beads A on which a double-stranded nucleic acid is formed.

Further, the magnetic beads to which the non-complementary capture probe is immobilized is also subjected to the same processing as above, thereby obtaining magnetic beads B on which no double-stranded nucleic acid is formed.

### (3) Electrochemical Measurement

After the above-mentioned processes, 5 μM of the magnetic beads A having the double-stranded nucleic acid and 5 μM of the magnetic beads B having no double-stranded nucleic acid are dropped onto the electrode, respectively. Thereafter, voltage is applied to the electrodes xA and yB on which the respective magnetic beads are consolidated, and electrochemiluminescence that occurs at this time is measured. This voltage application is carried out by scanning from 0V to 1.3V, and electrochemical measurement is carried out for three seconds. The measurement of electrochemiluminescence quantity is carried out using a photoelectron multiplier (H7360-01 produced by Hamamatsu Photonics), and the luminescence quantities obtained during the voltage scanning are integrated.

FIG. 2 is a diagram illustrating the electrochemiluminescence integral quantities which are detected on the electrode xA having the double-stranded nucleic acid and on the electrode yB having no double-stranded nucleic acid.

As is evident from FIG. 2, the luminescence quantity on the electrode xA having the double-stranded nucleic acid is significantly larger than the luminescence quantity on the electrode yB having no double-stranded nucleic acid, and it is discovered that the double-stranded nucleic acid, i.e., the target gene sample, can be detected with high sensitivity by using the method of the present invention.

### Example 3

### (1) Modification of Linker to Gene Sample

The same gene sample as that described for the first example is adopted. The gene sample adjusted to 100 μM is collected by 100 μL, and N-bromosuccinimide adjusted to 2 mM is added by 37.5 μL, and then the solution is gently mixed for 5 minutes while cooling the same with ice water.

After the agitation, the linker (chemical formula (11)) adjusted to 1 mM is added by 100 μL.

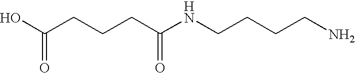

The linker represented by chemical formula (11) is obtained as follows.

Initially, 200 μL (2.00 mmol) of 1,4-butandiamine is dissolved in acetonitrile, and 537 mL (4.10 mmol) of triethylamine and 4 mg (1.00 mmol) of glutaric anhydride are added, and the solution is mixed for three hours at room temperature. Then, the crude product is purified with HPLC to obtain the linker represented by chemical formula (11) (yield 90.5%).

After the gene sample is thus modified with the linker, unreacted linker is removed using HPLC, and the solution is distilled by a centrifugal drying machine.

The following table shows the result of ¹H-NMR of the substance obtained as described above, which is represented by chemical formula (11).

¹H-NMR (300 MHz, CDCl₃)
σ:

1.43 (2H, m)
1.66 (4H, m)
2.10 (4H, m)
2.84 (2H, t)
3.06 (2H, m)
3.41 (2H, bs)

### (3) Hybridization

The same samples as those described in the first example are adopted as a capture probe and a non-complementary capture probe.

Initially, the above-mentioned linker bonded gene sample is adjusted to 2.0 μM by 2×SSC. Then, 2 μL of this solution, 3 μL of 0.1 μM capture probe, and 15 μL of 2×SSC are added to a micro tube, and the solution is gently mixed at 70° C. After mixing for one hour, the reaction solution is put in a dialysis tube, and dialyzed to desalt SSC.

In this third example, a non-complementary capture probe similar to that of the first example is used as a comparison target, and the same operation as that for the capture probe is carried out.

### (3) Addition of Electrochemically Active Substance

After the dialysis, 10 μL (20.0 μmol) of 2 μM WSC, 10 μL (2.0 μmol) of 0.2 μM N-hydroxysuccinimide, and 1.0 μL (1.0 pmol) of 1.0 μM electrochemically active substance (chemical formula (12)) are added, and the solution is gently mixed for one hour at room temperature.

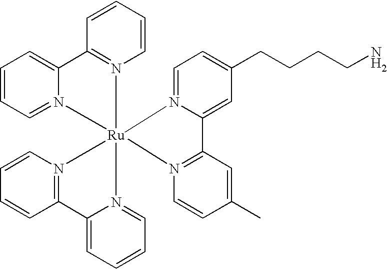

The electrochemically active substance represented by chemical formula (12) is obtained as follows.

Initially, 2.50 g (13.5 mmol) of 4,4′-dimethyl-2,2′bipyridine which is dissolved in 60.0 mL of THF is injected into a container under nitrogen atmosphere, and thereafter, 16.9 mL (27.0 mmol) of lithium diisopropylamide 2M solution is dropped, and the solution is stirred for 30 minutes while cooling the same. On the other hand, 4.2 mL (41.1 mmol) of 1,3-dibromopropane and 10 mL of THF are added in a container that is similarly dried in nitrogen gas stream, and the solution is stirred while cooling the same. The above-mentioned reaction solution is slowly dropped into this container, and reaction is promoted for 2.5 hours. The reaction solution is neutralized with 2N of hydrochloric acid, and the THF is distilled, and thereafter, the reactant is extracted with chloroform. Further, the crude product obtained by distilling the solvent is purified with silica gel column to obtain a product C (yield 47%).

Then, 1.0 g (3.28 mmol) of the product C, 0.67 g (3.61 mmol) of phthalimide potassium, and 30.0 mL of dimethylformamide (dehydrated) are added in a container under nitrogen atmosphere, and the solution is refluxed for eighteen hours in an oil bath. After reaction, the reactant is extracted with chloroform, and washed with distilled water using 50 mL of 0.2N sodium hydroxide. The solvent is distilled away, and recrystallization is performed by ethyl acetate and hexane, thereby obtaining a product D (yield 61.5%).

After ruthenium chloride (III) (2.98 g, 0.01 mol) and 2,2′-bipyridyl (3.44 g, 0.022 mol) are refluxed for six hours in dimethylformamide (80.0 mL), the solvent is distilled away. Thereafter, acetone is added, and the solution is cooled overnight to obtain a black precipitation. Thus obtained black precipitation is extracted, and 170 mL of ethanol aqueous solution (ethanol:water=1:1) is added, and the solution is heated and refluxed for one hour. After filtration, 20 g of lithium chloride is added, and ethanol is distilled away, and further, the solution is cooled overnight. The deposited black substance is collected by suction filtration, thereby obtaining a product E (yield 68.2%).

Then, 0.50 g (1.35 mmol) of the product D, 0.78 g (1.61 mmol) of the product E, and 50 mL of ethanol are added in a container that is nitrogen substituted. After this solution is refluxed for nine hours under nitrogen atmosphere, the solvent is distilled away, and the resultant is dissolved with distilled water and precipitated in 11.0M of perchloric acid solution. This precipitate is collected, and recrystallization is carried out with methanol, thereby obtaining a product F (yield 81.6%).

Furthermore, 11.0 g (1.02 mmol) of the product F and 70.0 mL of methanol are refluxed for one hour. After the solution is cooled down to room temperature, 0.21 mL (4.21 mmol) of hydrazine monohydrate is added, and the solution is again refluxed for thirteen hours. After reaction, 15 mL of distilled water is added, and methanol is distilled away.

Next, a reaction solution that is obtained by adding 5.0 mL of concentrated hydrochloric acid and performing refluxing for two hours is cold-stored overnight, and impurities are removed by normal filtration.

After this solution is neutralized with sodium hydrogen carbonate, water is distilled away, and inorganic substances are removed with acetonitrile. The crude product obtained by distilling the solvent away is purified with silica gel column, thereby obtaining the electrochemically active substance represented by chemical formula (12) (yield 71.4%).

The following table shows the result of ¹H-NMR of the substance obtained as described above, which is represented by chemical formula (12).

¹H-NMR (300 MHz, DMSO d-6)
σ:

1.68 (4H, m)
2.52 (3H, s)
2.84 (4H, m)
3.40 (2H, bs)
7.38 (2H, d)
7.58 (6H, m)
7.73 (4H, m)
8.15 (4H, t)
8.76 (2H, d)
8.86 (4H, d)

After the electrochemically active substance is bonded to the gene sample as described above, 20 μL of magnetic beads similar to those of the second embodiment are added, and the solution is gently mixed for one hour, whereby the capture probe is immobilized to the magnetic beads to obtain magnetic beads C on which a double-stranded nucleic acid is formed.

Further, a non-complementary capture probe is also immobilized to magnetic beads in similar manner as described above to obtain magnetic beads D on which no double-stranded nucleic acid is formed.

### (4) Electrochemical Measurement

After the above-mentioned processes, the magnetic beads C having the double-stranded nucleic acid and the magnetic beads D having no double-stranded nucleic acid are dropped each by 5 μL onto the electrode. A permanent magnet sheet is attached beneath the electrode so as to gather the magnetic beads to only the working electrode.

After still standing for five minutes, 75 μL of electrolytic solution is dropped onto the electrode xC and yD on which the magnetic beads C and D are gathered, respectively. Thereafter, voltage is applied to the respective electrodes xC and yD on which the respective magnetic beads are gathered, and electrochemiluminescence which occurs at this time is measured. This voltage application is performed by scanning from 0V to 1.3V, and electrochemical measurement is carried out for three seconds. Measurement of electrochemiluminescence quantity is carried out using a photoelectron multiplier (H7360-01 produced by Hamamatsu Photonics), and the luminescence quantities obtained during the voltage scanning are integrated.

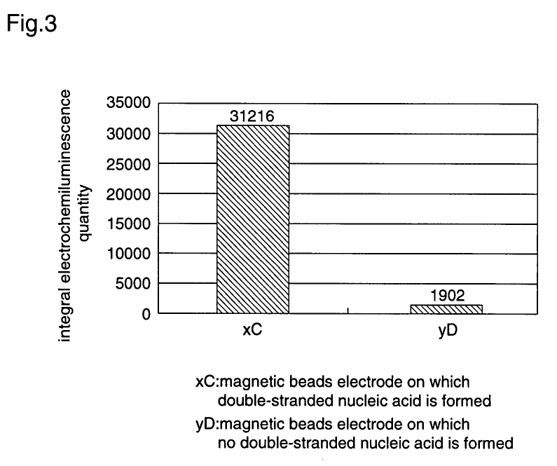

FIG. 3 is a diagram illustrating the integral electrochemiluminescence quantities which are detected from the electrode xC of the magnetic beads on which the double-stranded nucleic acid is formed and from the electrode yD of the magnetic beads on which no double-stranded nucleic acid is formed.

As is evident from FIG. 3, the luminescence quantity on the magnetic beads electrode xC having the double-stranded nucleic acid is significantly larger than the luminescence quantity on the magnetic beads electrode yD having no double-stranded nucleic acid, and it is discovered that the double-stranded nucleic acid, i.e., the target gene sample, can be detected with high sensitivity by using the method of the present invention.

### APPLICABILITY IN INDUSTRY

A gene detection method according to the present invention can detects a gene having a specific sequence with high sensitivity, and it is applicable to genetic testing, infection testing, genome-based drag discovery, and the like.

1. A gene detection method for detecting a gene having a specific sequence in a test sample, said method comprising:
an immobilization step of forming a single-stranded capture probe having a base sequence that is complementary to the target gene to be detected, and immobilizing the capture probe to a solid phase;
a gene sample formation step of forming a gene sample by denaturing the target gene into a single strand;
a bonding step of chemical bonding the gene sample and an electrochemically active substance;
a gene sample capturing process of hybridizing the gene sample to which the electrochemically active substance is bonded, with the single-stranded capture probe that is immobilized to the solid phase, thereby to make the solid phase capture the gene sample; and
a detection step of detecting the electrochemically active substance that is bonded to the gene sample immobilized to the solid phase, by electrochemical measurement.

2. A gene detection method for detecting a gene having a specific sequence in a test sample, said method comprising:
a gene sample formation step of forming a gene sample by denaturing the target gene to be detected into a single strand;
a bonding step of chemical bonding the gene sample and a linker having a site that binds to an electrochemically active substance;
a double-stranded nucleic acid formation step of forming a single-stranded capture probe having a base sequence that is complementary to the target gene, and hybridizing the capture probe with the gene sample to which the linker is bonded, thereby forming a double-stranded nucleic acid;
a reaction step of chemical bonding the electrochemically active substance and the linker of the gene sample in which the double-stranded nucleic acid is formed;
an immobilization step of immobilizing the capture probe in which the double-stranded nucleic acid is formed, to a solid phase; and
a detection step of detecting the electrochemically active substance that is bonded to the gene sample immobilized to the solid phase, by electrochemical measurement.

3. A gene detection method for detecting a gene having a specific sequence in a test sample, said method comprising:
an immobilization step of forming a single-stranded capture probe having a base sequence that is complementary to the target gene to be detected, and immobilizing the capture probe to a solid phase;
a gene sample formation step of forming a gene sample by denaturing the target gene into a single strand;
a bonding step of chemical bonding the gene sample and a linker having a site that binds to an electrochemically active substance;
a gene sample capturing process of hybridizing the gene sample to which the linker is bonded with the single-stranded capture probe that is immobilized to the solid phase, thereby to make the solid phase capture the gene sample to which the linker is bonded;
a reaction step of adding the electrochemically active substance to the gene sample that is captured by the solid phase, thereby chemical bonding the linker that is bonded to the gene sample, to the electrochemically active substance; and
a detection step of detecting the electrochemically active substance that is bonded to the gene sample immobilized to the solid phase, by electrochemical measurement.

4. A gene detection method for detecting a gene having a specific sequence in a test sample, said method comprising:
a gene sample formation step of forming a gene sample by denaturing the target gene to be detected into a single strand;
a bonding step of chemical bonding the gene sample and an electrochemically active substance;
a double-stranded nucleic acid formation step of forming a single-stranded capture probe having a base sequence that is complementary to the target gene, and hybridizing the capture probe with the gene sample to which the electrochemically active substance is bonded, thereby forming a double-stranded nucleic acid;
an immobilization step of immobilizing the capture probe in which the double-stranded nucleic acid is formed, to a solid phase; and
a detection step of detecting the electrochemically active substance that is bonded to the gene sample immobilized to the solid phase, by electrochemical measurement.

5. A gene detection method as defined in claim 1 or 4 wherein said bonding step of bonding the gene sample and the electrochemically active substance is carried out, after a halogen compound is added to bases in the gene sample, by promoting a nucleophilic substitution reaction between a functional group in the electrochemically active substance and the halogen that is bonded to the bases in the gene sample.

6. A gene detection method as defined in claim 2 or 3 wherein said bonding step of bonding the gene sample and the linker is carried out, after a halogen compound is added to bases in the gene sample, by promoting a nucleophilic substitution reaction between a functional group in the linker and the halogen that is bonded to the bases in the gene sample.

7. A gene detection method as defined in claim 1 or 4 wherein said electrochemically active substance is represented by chemical formula (1) as follows:
NuLa)m-E
wherein Nu is nucleophile selected from among amine group, alcohol group, ether group, thiol group, and oxide group, E is an electrochemically active site, and La is a connection site that connects the Nu to the E.

8. A gene detection method as defined in claim 2 or 3 wherein said linker is represented by chemical formula (2) as follows:
NuLb)n-E
wherein Nu is a nucleophile selected from among amine group, alcohol group, ether group, thiol group, and oxide group, Sa is a site that chemical binds to the electrochemically active substance, and Lb is a connection site that connects the Nu to the Sa.

9. A gene detection method as defined in claim 2 or 3 wherein said electrochemically active substance is represented by chemical formula (3) as follows:
NuLc)o-E
wherein E is an electrochemically active site, Sb is a site that chemically bonds to the Sa, and Lc is a connection site that connects the Sb to the E.

10. A gene detection method as defined in any of claims 7 to 9 wherein said La, Lb, and Lc are substances selected from among alkyl, alcohol, carboxylic acid, sulfo acid, ester, ketone, thiol, ether, amine, nitro, nitrile, sugar, phosphate, amino acid, methacrylic acid, amide, imide, isoprene, urethane, uronic acid, ethylene, carbonate, vinyl, cycloalkane, and heterocyclic compound, and a combination of some of these substances.

11. A gene detection method as defined in claim 8 or 9 wherein the chemical bonding between the Sa and the Sb is one selected from among amide bonding, ester bonding, ether bonding, thioether bonding, sulfide bonding, carbonyl bonding, imino bonding, and antibody-antigen bonding.

12. A gene detection method as defined in claim 7 wherein the m is an integer ranging from 4 to 50.

13. A gene detection method as defined in claim 8 wherein the n is an integer ranging from 1 to 50.

14. A gene detection method as defined in claim 9 wherein the o is an integer ranging from 1 to 1000.

15. A gene detection method as defined in claim 14 wherein the o is an integer ranging from 3 to 1000 when the n is 1, and the o is an integer ranging from 2 to 1000 when the n is 2.

16. A gene detection method as defined in claim 7 or 9 wherein the E is a compound having oxidation-reduction property.

17. A gene detection method as defined in claim 16 wherein the compound having oxidation-reduction property is a compound which exhibits electrochemiluminescence.

18. A gene detection method as defined in claim 17 wherein the compound which exhibits electrochemiluminescence is one selected from among a metal complex having a heterocyclic compound as a ligand, rubrene, anthracene, coronene, pyrene, fluoranthene, chrysene, phenanthrene, perylene, binaphthyl, and octatetraene.

19. A gene detection method as defined in claim 18 wherein the metal complex having a heterocyclic compound as a ligand is a metal complex having a pyridine site as a ligand.

20. A gene detection method as defined in claim 19 wherein the metal complex having a pyridine site as a ligand is one of a metal bipyridine complex and a metal phenanthroline complex.

21. A gene detection method as defined in claim 20 wherein a center metal of the metal complex having a heterocyclic compound as a ligand is one of ruthenium and osnium.

22. A gene detection method as defined in any of claims 1 to 4 wherein said detection step includes applying a voltage to the solid phase, and measuring the quantity of electrochemiluminescence from the linked electrochemically active substance.


SEQUENCE LISTING (3 sequences)

SEQ ID NO: 1 - DNA, 30 bp, Homo sapiens
  misc_feature: capture probe
aatttgttatgggttcccgggaaataatca

SEQ ID NO: 2 - DNA, 100 bp, Homo sapiens
  misc_feature: gene sample
aattgaatgaaaacatcaggattgtaagcaccccctggatccagatatgcaataattttcccactatcattgattatttcccgggaacccataacaaatt

SEQ ID NO: 3 - DNA, 30 bp, Artificial Sequence
  synthetic oligonucleotide
aaaaaaaaaaaaaaaaaaaaaaaaaaaaaa